(19) United States
(12) Patent Application Publication
(10) Pub. No.: US 2019/0060099 A1
(43) Pub. Date: Feb. 28, 2019

(54) WEARABLE AND FUNCTIONAL HAND ORTHOTIC

(71) Applicant: THE TRUSTEES OF COLUMBIA UNIVERSITY IN THE CITY OF NEW YORK, NEW YORK, NY (US)

(72) Inventors: Matei Ciocarlic, New York, NY (US); Joel Stein, Sharon, MA (US)

(21) Appl. No.: 15/766,897

(22) PCT Filed: Oct. 7, 2016
(86) PCT No.: PCT/US16/55969

§ 371 Date: Apr. 9, 2018

Related U.S. Application Data
(60) Provisional application No. 62/239,802, filed on Oct. 9, 2015.

(51) Int. Cl. A61F 5/01; A61H 1/02; A41D 19/00

(52) U.S. Cl. CPC A61F 5/0118; A61H 1/0288; A41D 19/00; A61F 2005/0155; A41D 2400/32; A61H 2201/14; A61H 2201/1635; A61H 2201/165; A61H 2230/085; A61H 2201/1207

(57) ABSTRACT

A motor-driven wearable hand orthotic.

### CROSS REFERENCE TO RELATED APPLICATIONS

This application claims priority to U.S. Provisional Patent Application Ser. No. 62/239,802, filed Oct. 9, 2015, the content of which is hereby incorporated by reference in its entirety.

### FIELD

The present disclosure relates to a wearable hand orthotic for regaining movement in stroke victims and/or rehabilitation for occupational therapy patients.

### BACKGROUND

A stroke occurs when reduced blood supply to the brain or bleeding within the brain causes brain cells to die. Approximately 800,000 people in the United States have a stroke each year. Injury to the portion of the brain that controls motor function can result in weakness or paralysis of the arms and/or legs due to loss of muscle function. Paralysis of the limbs can also be caused by brain trauma, spinal cord injury, nerve or muscle diseases, or autoimmune diseases.

Patients who survive a stroke must undergo rehabilitation to regain function of an impaired limb, however studies have shown that over half of the subjects still require assistance with daily tasks even after rehabilitation. Many post-stroke patients suffer from weakened or paralyzed muscles resulting from injury to the motor cortex of the brain. Studies have shown that rehabilitation alone may not be able to help the majority of patients regain enough functional control of an impaired hand and/or arm to independently perform daily activities.

Upper extremity orthoses are external devices designed to help restore musculoskeletal and nervous system function. Advanced mechanical and electrical engineering have led to the development of dynamic/functional orthoses that assist the movement of weak muscles and help increase functional ability.

There is a need for a comfortable, wearable, light-weight, functional hand orthotic capable of generating the amount of force necessary to assist in hand movement.

### SUMMARY

The disclosed subject matter generally provides a wearable hand orthotic comprising a glove, a network of exotendons and a single actuator that is capable of actuating multiple exotendons. In one embodiment, the wearable functional hand orthotic comprises a flexible glove having a dorsal surface and a palmar surface and a plurality of sheaths for receiving digits of a hand. A network of exotendons is disposed on one or both surfaces of the glove. The network of exotendons includes a plurality of exotendons. Each exotendon has a longitudinal body including a distal end and a proximal end. The longitudinal body is routed along and secured to the glove, and in particular the sheaths in which digits will be received when worn. The network of exotendons is operatively engaged to an actuator. The actuator is capable of actuating the plurality of exotendons to promote movement of the hand in various different motions according to the routing configuration of the exotendons. In some embodiments the wearable hand orthotic comprises both a glove and a sleeve. The actuator, e.g., motor, can be disposed on the glove or the sleeve. In embodiments where the motor is disposed on the sleeve, the exotendons may extend from the glove to the sleeve, e.g., fingers to the elbow.

Suitable actuators include small form-factor motors capable of generating the required levels of force to support hand movements. For example, the actuator may be light-weight, weighing about 40 grams. In operation, the actuators drive the artificial tendons to provide assistance with hand movement and force generation. Accordingly, the wearable orthotic facilitates the execution of bi-manual tasks (i.e. one hand provides support while the other performs fine movements). This allows users to regain use of the hand in order to independently perform daily tasks.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 depicts one embodiment of the wearable orthotic.

FIG. 2 depicts another embodiment of the wearable orthotic comprising a glove including a sleeve.

FIG. 3 depicts an embodiment of the wearable orthotic having exotendons disposed on the dorsal and palmar surfaces of the glove.

FIG. 4 depicts the palmar view of the glove of FIG. 3.

FIG. 5(a) to (d) depict various routing configurations for the exotendons in accordance with the wearable orthotic.

FIG. 6 shows photographs depicting a routing configuration for the exotendon in accordance with the wearable orthotic.

FIG. 7 shows an exotendon device including an EMG armband which provides control input.

FIG. 8 shows force results for an experimental subject using an embodiment of the wearable orthotic.

FIG. 9 shows force results for another experimental subject using an embodiment of the wearable orthotic.

FIG. 10 are graphs comparing force measurements by button control and EMG control.

### DETAILED DESCRIPTION OF EXEMPLARY EMBODIMENTS

The disclosed subject matter generally provides a compact wearable hand orthotic comprising a fabric glove. The fabric is routed with a network of artificial tendons that are connected to one or more lightweight linear actuators. These actuators generate enough force to drive the artificial tendons to provide assistance with hand movement. The orthotic is useful for rehabilitation robotics for patients suffering from post-stroke effects and motor neuron diseases, as well as suppression of tremors arising from neurological disorders such as Parkinson's disease, correction of abnormal hand posture and conditions such as Swan-Neck deformity, sports medicine, strength training, and other applications.

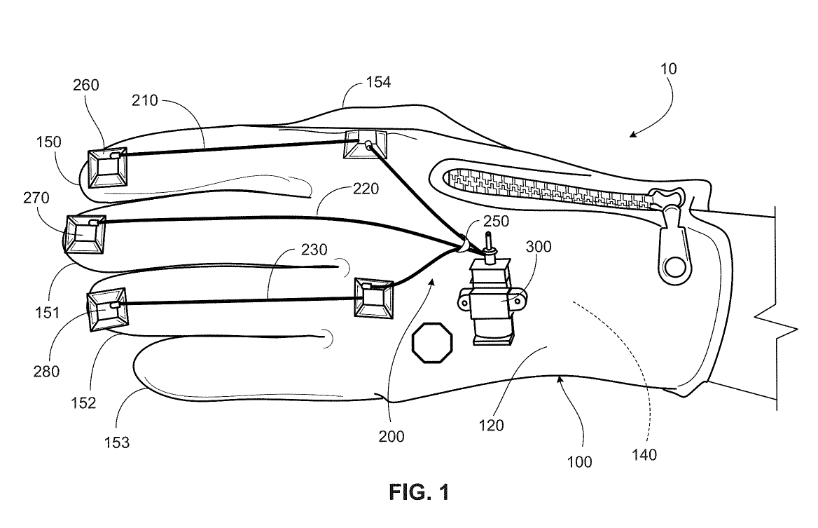

The wearable, functional, orthotic according to one embodiment is illustrated in FIG. 1. The wearable functional orthotic 10 generally includes a glove 100 having a dorsal surface 120 and a palmar surface 140 and a plurality of sheaths 150, 151, 152, 153, 154 for receiving the digits of a person's hand. The glove is preferably formed from a comfortable, lightweight, washable, fabric. A network of exotendons 200 is disposed on a surface of the glove 100. The exotendons are formed from bendable, flexible material, such as but not limited to, braided polyethylene fiber (commonly used in modern fishing lines). Diameters and durometers of such wire can vary; in one instantiation we use 0.018 in diameter wire with a rated strength of 100 lbs. The network of exotendons 200 comprise a plurality of exotendons 210, 220, 230 routed along the sheaths 150, 151, 152 such that each exotendon has a corresponding sheath and digit when worn. The exotendons are routed through guides 260, 270, 280 fastened to the glove. The guide directs the pathway of the exotendon along the individual sheaths. Suitable guides include a ringlet, eye, hook, and the like. The guides may be detachably connected to the glove so that the glove can be detached and washed. The proximal ends of each exotendon lead to junction 250. The junction is proximate and operative connected to an actuator, e.g., motor 300. The actuator initiates movement of the exotendons such that the digits of the hand are moved when the glove is in operation while worn. The actuator is capable of actuating multiple exotendons. Suitable actuators include, but are not limited to, electric motors and pneumatic actuators. Suitable actuators are small and lightweight (about 40 g). This includes electric motors with transmission producing linear motion (e.g. Firgelli L12 and L16, weighing 40 g and 50 g respectively) or rotary motion (e.g. Pololu Micro Metal Gearmotor, 20D Metal Gearmotor and 25D Metal Gearmotor, weighing 10 g, 42 g, and 82 g respectively). As shown, the motor 300 can be disposed on the glove 100 to provide hand-mounted actuation. FIG. 1 exemplifies a rotary motor mounted on the dorsal side of hand and illustrates for example an extensor extending network with three branches for three digits.

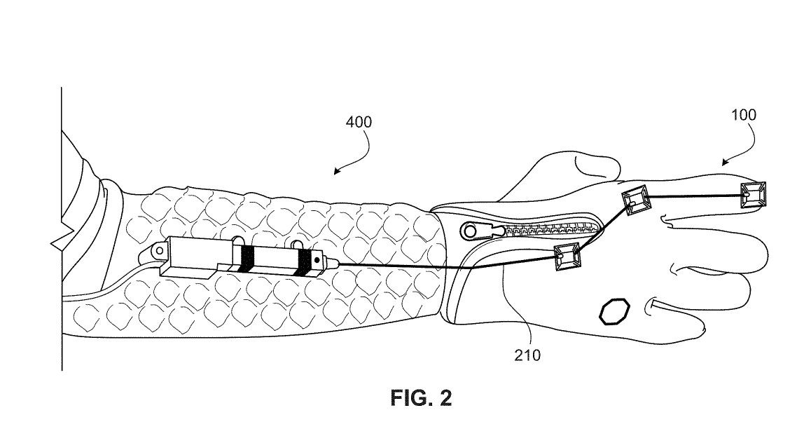

As shown in FIG. 2, the wearable functional orthotic may further include a sleeve 400. FIG. 2 shows the orthotic having a glove 100 and a sleeve 400 on which a single exotendon 210 routed along the digit and the hand to forearm for forearm mounted actuation.

In accordance with another embodiment, a wearable functional orthotic having a plurality of exotendons disposed along both the dorsal and palmar surfaces of the glove is provided.

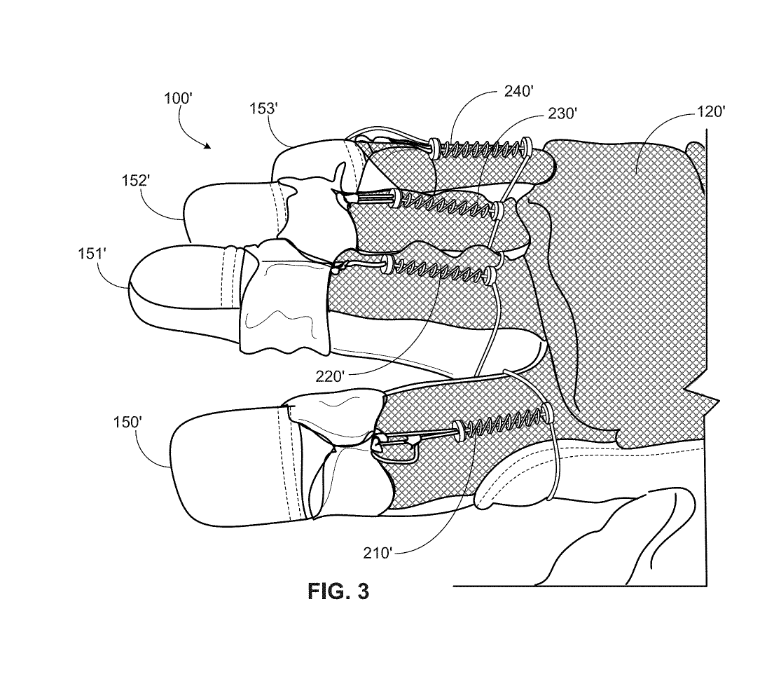

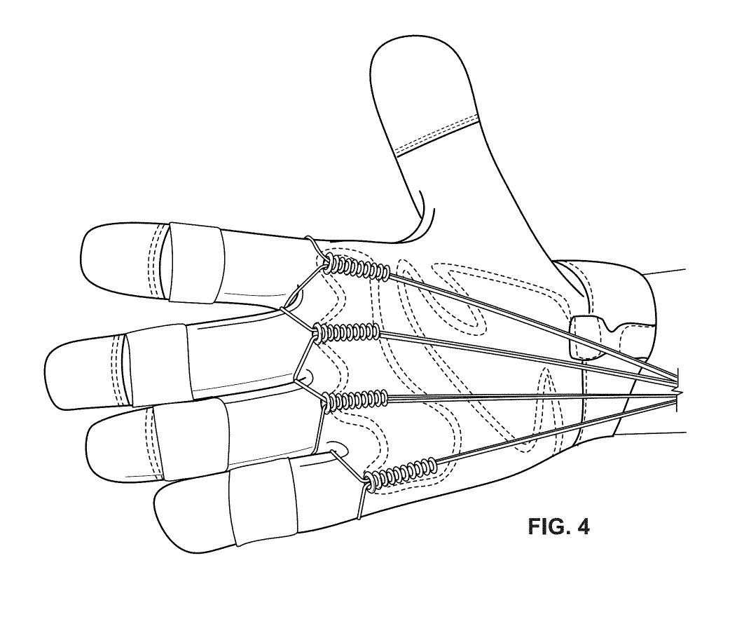

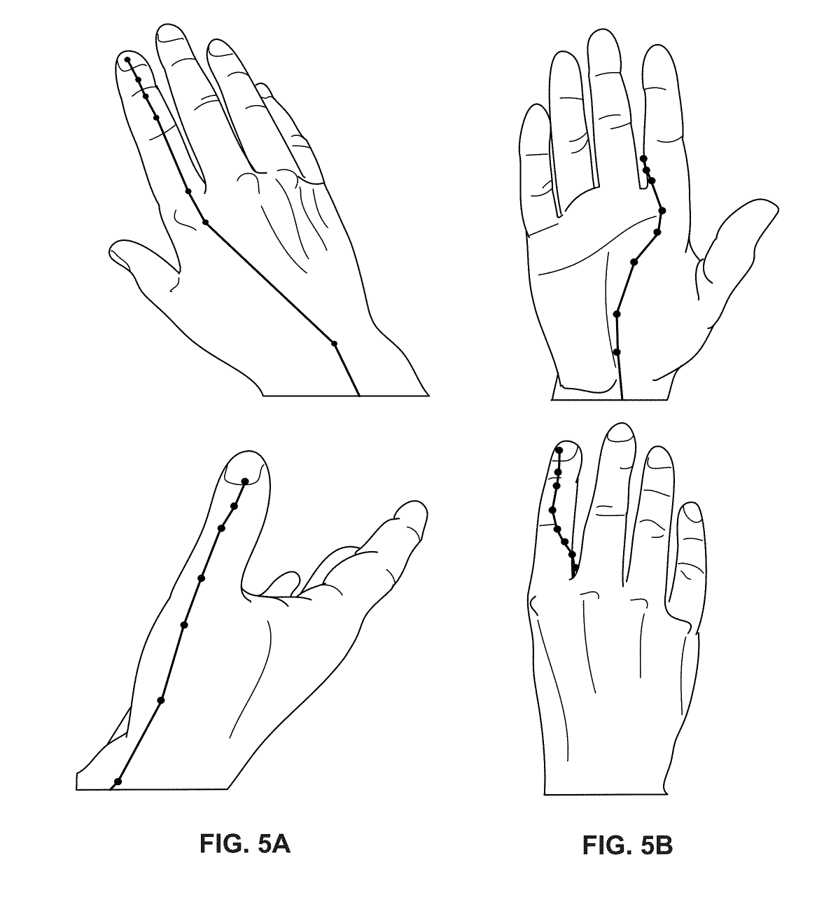

Referring to FIG. 3, the dorsal surface 120′ of wearable functional orthotic 100′ is shown. A first plurality of exotendons 210′, 220′, 230′, 240′ is routed and disposed along the sheaths 150′, 151′, 152′, 153′ as depicted. As best shown in FIG. 4, the plurality of exotendons are routed about the sheath and traverse the glove to the palmar side of the glove to form a second plurality of exotendons traversing the palm portion of the glove and defining a second plurality of exotendons. The first plurality of exotendons is operatively attached to or alternatively, integrally define, a second plurality of exotendons routed from the proximal portion of the sheath and proximally along the metacarpal section of the glove on the palmar surface. In this embodiment, the exotendons are routed along both the palmar and dorsal sides of the glove and the hand of the subject wearing the glove. In this embodiment, the longitudinal bodies of the exotendons are routed on the palmar side of the glove and flexion side of a metacarpophalangeal joint of the user. The longitudinal bodies of the exotendons traverse to the dorsal side of the proximal interphalangeal and distal interphalangeal joints of the user. When actuated, this embodiment enables a fingertip pinch grasp. Accordingly, the exotendons disposed on the dorsal and palmar surfaces of the glove are actuated by motors and provide the wearer with the ability to grasp, hold, and pinch objects.

The wearable functional orthotic may be modified with various different routing schemes for the network of exotendons, depending on desired hand function. Tendon route determination for the embodiments of the orthotic embodied herein depends on desired joint co-actuation pattern. Certain patterns of impairments are particularly common among stroke survivors, and remain challenging from a rehabilitation perspective.

We note however that there are a wide range of motor and functional impairments that affect stroke survivors, and these are only intended as examples of common patterns to provide targets for device development. In one pattern, individuals are able to form a gross grasp with the hand moving all digits in synergy but lack the ability for sufficient finger extension to actively open the hand after grasping. These individuals also typically lack individuated finger movements. The functional use of the hand in this situation may be completely absent or limited to grasping an object while it is manipulated by the other hand (e.g, a tube of toothpaste). This pattern corresponds with stage 3 score on the Chedoke-McMaster Stroke Assessment Test.

Another common pattern is the ability to move all digits of the hand, but to have limited individuation, and for the movements to be slow, lacking in dexterity, and of diminished force. Such an individual may be able to oppose the thumb to each of the other digits in sequence, but only slowly and with considerable effort. The ability to manipulate objects is limited, and the use of a computer keyboard, buttons, and shoelaces not feasible. This pattern corresponds with a stage 6 score on the Chedoke-McMaster Stroke Assessment test.

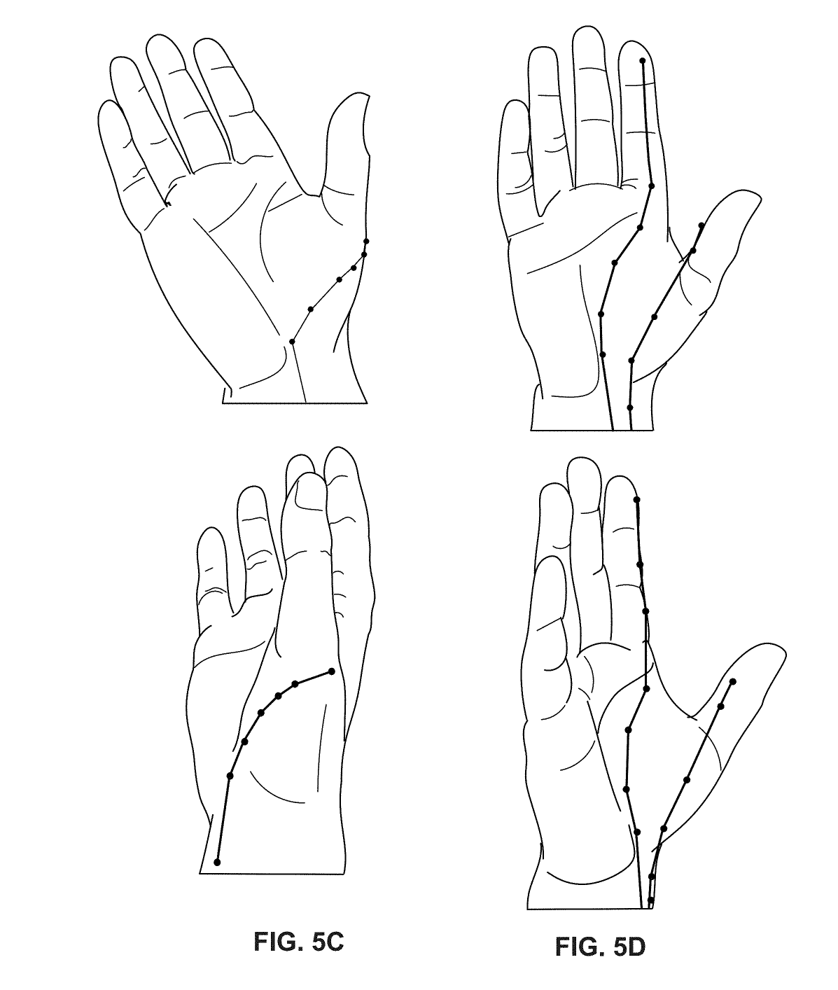

Referring to FIGS. 5(a) to 5(d), the orthotic described and embodied herein can be constructed with various different exotendon routing for hand extension to address help individuals overcome issues with the patterns described above. The co-actuation pattern offered by the orthotic described and claimed can be driven by a network of the exotendons. Combined extension of all digits is promising since it is consistent with the first synergy observed for human grasping, which accounts for more than 50% of hand posture variance. Combined finger extension is amenable to direct implementation through a single motor, since a tendon can be routed on the dorsal side of all joints all the way to the distal phalanxes.

FIG. 5 (a) shows an example extensor tendon branch for the index finger; the physical implementation of this route is illustrated in FIG. 2. Branches for the middle, ring and little fingers, originating from the same main tendon, can follow similar routes. These routes also allow the tendons to be neutral with regard to abduction/adduction motion of the fingers. In this embodiment, complete flexion of all the joints of the index finger requires about 57 mm of travel of the tendon, matching the specifications of the linear actuators shown in FIG. 2. FIG. 5a also shows a branch of the extensor tendon for the thumb. Thetrapeziometacarpal (TMC) joint is significantly more complex than finger carpometacarpal (CMC) joints; in this embodiment a route having two non-perpendicular axes of rotation is used. In one embodiment of the orthotic, the extensor tendon has a limited but non-zero effect on thumb abduction. However, this effect is also consistent with identified human synergies. Full flexion of the thumb requires about 52 mm of travel of the tendon.

FIG. 5(b) shows a second configuration: metacarpophalangeal (MCP) flexion/interphalangeal (IP) extension. This exemplified routing pattern assumes opposite motion at the MCP joints versus the IP joints for each finger. For the thumb, the role of MCP flexion is instead played by adduction. This is the dominant pattern of the second most important synergy found in human hand grasp postures. Functionally, it allows transition between enveloping postures and fingertip opposition postures. For stroke patients exhibiting the ability to move all digits of the hand, but have limited individuation, and slow movements, lack in dexterity, and diminished force, this configuration could increase the range of grasps that can be executed. From an implementation perspective, extension at a finger IP joints can be achieved with an exotendon routed on the dorsal side of the finger; however, that route also extends the MCP joint (as in the previous configuration), an effect that is undesirable here. One way to mitigate that effect is to provide an additional exotendon routed on the palmar side of the MCP joint to provide an antagonistic effect. Alternatively, a single exotendon can be routed on the palmar side of the MCP joint then wrap around the finger to the dorsal side of the proximal and distal IP joints, as shown in FIG. 5b. This routing configuration also has an effect on finger adduction, which is consistent with observed human synergies and unlikely to adversely affect grasps using the fingertips. This configuration could be implemented for only the thumb and the index finger, enabling a two-finger pinch, or for all digits, enabling multiple grasping configurations for the entire hand.

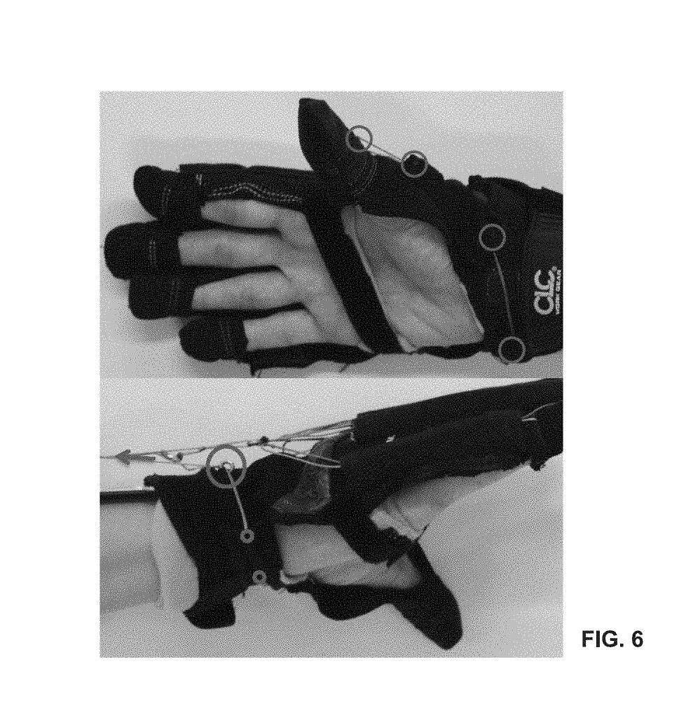

FIG. 5(c) depicts a configuration for thumb opposition/abduction. Assistance placing the thumb in opposition to the other fingers is needed for many grasp types and manipulation tasks, while thumb abduction is a part of opening the hand before grasping. The exotendon routing of FIG. 5(c) shows a proposed exotendon configuration for thumb opposition and abduction. Tendon displacement for full TMC joint range of motion is 28 mm, allowing for shorter stroke motors thumb, and does not have a direct equivalent in the list of observed dominant human synergies (which have a non-zero component for most hand joints). Other configuration are also suitable for the orthotic, such as shown in FIG. 5(d) which assist finger flexion, individuating the index finger or the index and middle finger pair, etc. Each of the configurations described and shown in FIGS. 5(a) to (d) may be used alone in an orthotic equipped with a single motor. The routing configurations may also be combined in one orthotic for higher dexterity. FIG. 6 depicts images of the glove having yet another routing configuration, which includes a route for the thumb tendon. The exotendon traverses down the dorsal side of the thumb, but then traverses the wrist on the palmar side and wraps around the arm to the other side. The goal in this routing configuration is to bring the thumb in opposition to the other fingers at the same time as opening it. The circles in FIG. 6 depict attachment points of the exotendon to the glove.

In another aspect, two different approaches for interfacing between the user and the orthotic are provided. The upper limb weakness after stroke is typically unilateral (hemiparesis). As such, in one approach the voluntary motion of the less affected limb is used as an input signal for the orthotic used on the more affected limb. In such cases, the wearer of the orthotic uses the less affected limb exclusively for tasks requiring the use of a single hand. This type of bimanual operation uses natural movement as an input signal and takes advantage of the non-symmetric nature of impairment.

In a unimanual approach, the orthotic assists the wearer's voluntary commands sent directly to the paretic hand. If, despite the impairment, the patient can generate noticeable endogenous movements, these could be detected by IMU devices embedded in the orthotic along the exotendon network. Alternatively, a subset of the tendons can be connected to displacement measurement sensors rather than to a motor and serve as joint position and movement sensors.

Motors may be equipped with force sensors on output so the tendon network can also serves the role of joint torque sensing, and can thus detect voluntary joint commands. One method for using this approach involves wearer's with involuntary finger flexion: if a voluntary extension command produces movement that is detectable but insufficient to open the hand for

grasping, the orthosis (using for example exotendon configuration shown in FIG. 2) could engage to assist extension, then release to allow the hand to close on an object.

In accordance with another aspect, EMG can be used to detect commands sent to the extrinsic hand muscles located in the forearm. Evidence shows signals exist and can be detected in stroke victims, but the muscle does not generate enough force to perform the desired motion (for example, the digit extensors are unable to overcome involuntary contraction of the flexors). The wearable orthotic may be designed to include a forearm sleeve to house actuators and surface EMG sensors (combining electrodes and amplifies) under or within the sleeve. Onset detection can be used to trigger binary commands placing the hand in a pre-defined pose. This method can trigger hand opening, allowing the patient to subsequently close the hand on the object. Conversely, proportional control based on the strength of contraction can be used for the position set point to the actuators, thus controlling for example the aperture of the hand in the open pose.

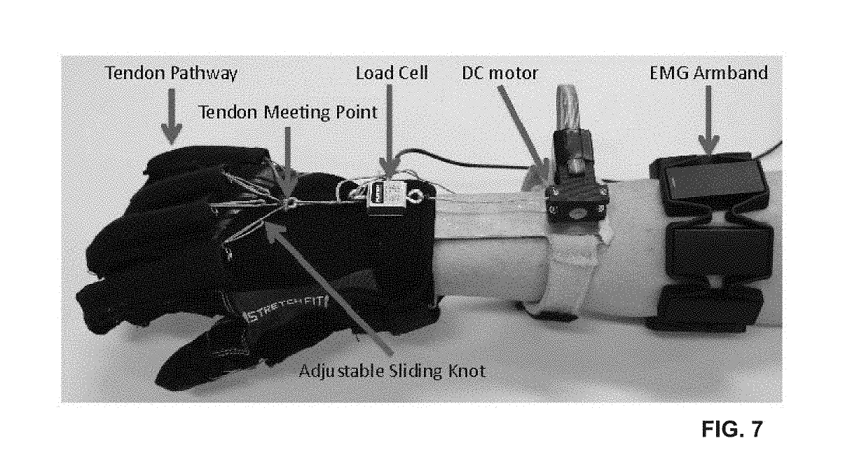

Accordingly, in another embodiment a system is provided that includes the exotendon device described above, and a control mechanism based on surface EMG. As shown in FIG. 7, the system includes a glove having an exotendon pathway, as described above with respect to the routing configurations, a load cell, DC motor and EMG armband. The EMG armband provides control input. This approach may use pattern classification—identifying patterns in EMG signals from the entire forearm to determine user intention.

In operation, EMG pattern classification is provided to determine the user's intention either to open or close the hand. This signal is used to produce physical movement assisted by the orthosis. Thus, the forearm EMG-based control apparatus and method can determine the user's intention to execute specific hand movements and exhibits a number of desirable characteristics for wearable devices. This embodiment includes an easy to don, multi-sensor device that does not require placing sensor son specific muscles and only needs short training time.

The control signal can be used in conjunction with an exotendon device to physically elicit the desired movement patterns in stroke patients. The use of the EMG driven orthosis in a functional context for picking and placing tasks whether the orthosis is entirely under the user's control is provided.

In one embodiment, an EMG controlled exoskeleton hand orthosis for stroke patients is provided. This is a step toward creating a user-controlled take home orthotic device that can help perform functional tasks. As described above, the subject matter relates to an exotendon orthosis comprising a soft glove with guided tendons driven by linear electric actuators to elicit desired movement patterns. In another aspect, a control mechanism for the exotendon device based on surface EMG is described. FIG. 7 shows an exotendon device, including an EMG armband which provides control input. With regard to this embodiment, the approach employs pattern classification—identifying patterns in EMG signals from the entire forearm—to determine user intention. EMG pattern classification is applied to determine the user's intention either to open or to close the hand. This signal is used to produce physical movement assisted by the orthosis. Accordingly, the subject matter described includes a forearm EMG-based control method that can determine the user's intention to execute specific hand movements, and that exhibits a number of desirable characteristics for wearable devices. This approach uses an easy to don, multi-sensor device that does not require placing sensors on specific muscles, and only needs a short training time.

The control signal described above can be used in conjunction with an exotendon device to physically elicit the desired movement patterns in stroke patients. Furthermore, the use of the EMG-driven orthosis in a functional context allows for pick-and-place tasks where the orthosis is entirely under the user's control. Described herein is a complete device, combining pattern classification with physical assistance. It has been found that it is important to develop and study these components together, as the presence of the physical device alters the EMG data obtained during operation. Steps to mitigate this phenomenon, ultimately enabling user-driven execution of complete tasks are described supra.

The EMG-based control method is implemented and tested on a complete hand orthosis device. A five fingered exotendon robotic device was used in the experiments, which assisted with hand extension; often a difficult task for stroke patients because of the commonly observed impairment pattern of spasticity, which is excessive involuntary hand flexion. Hand extension is achieved by applying extension torques on the fingers through an exotendon network pulled by a DC motor. The mechanical components of the device included two modules: a forearm piece with actuation and a glove with a tendon network. The two modules are connected via eye rings on both sides of the load cell to facilitate the donning process, as shown in FIG. 7.

Referring to FIG. 7, the forearm piece is composed of an aluminum splint and a DC motor. The splint constrains wrist movement to efficiently transmit the external torques to the fingers. The splint is angled either at 30 degrees, considered functional wrist pose or at 0 degrees for the patients who could not extend the wrist due to spasticity. A DC motor (Pololu corporation, 210:1 Low-Power Micro Metal Gearmotors) with a 15 mm/s maximum travel speed and a 80N peak force is mounted on the splint. Motor specifications are chosen to prevent dangerous tendon force levels without taking up space to improve wearability. A Proportional-Integral-Derivative (PID) position control is implemented to drive the motor. The motor's range of motion is determined at the beginning of clinical tests, depending on hand size.

The glove with a tendon network has tendons guided from the heads of the middle phalanges through raised pathways to a meeting point on the back of the hand. The tendons on each finger are attached to a cloth ring on the middle phalanges rather than on the fingertips to avoid finger hyperextension. The tendons on all digits, except for the thumb, are routed on the dorsal side of the glove. The thumb needs a special routing scheme since it exhibits different movement patterns from the other digits; the thumb tendon is routed from the proximal phalanx head to the metacarpal joint, then wraps clockwise around the wrist to the eye ring on the load cell. The tendons on all digits are tied with sliding adjustable knots to allow better fit for different finger lengths.

The DC motor which pulls the exotendon network is driven by the EMG-based control. Once the EMG control determines the user's intention to open or close the hand, it sends a command to the DC motor mounted on the splint. The motor then extends or retracts the tendon network to allow the user to open or close the hand.

EMG patterns of the hemiparetic forearm are often altered after a stroke event. This experiment was based on the assumption that these altered EMG patterns can still be used to control a hand orthosis; as control using forearm EMG sensors has a number of compelling characteristics. EMG-based control requires the same type of muscle activation as pre-stroke extension, which should make the control intuitive and place a low cognitive load on the user. Additionally, using ipsilateral EMG control leaves the other hand free to participate in the grasping task or to perform a different task.

Beyond altered signals however, EMG control of an orthosis for a stroke patient is also difficult because of additional phenomena, such as spasticity and abnormal coactivation relationships between muscles. As such, many orthoses that enable pick and place collect signals from only two muscles, with each muscle controlling a direction of the orthosis, often using a threshold based on the subject's maximum voluntary contraction. In these approaches, the subject must fully extend or close before the orthosis will move in the other direction. The user's ability to end extension allows more natural grasping for smaller objects as well as the option to change grasping tasks mid-motion.

One of the tenets of this method relies on signals from a multitude of sensors, placed around the circumference of the forearm. Instead of simple intensity thresholding, which is effective for a single sensor precisely located on a specific muscle, pattern classification was used to identify patterns in the complete set of signals from the sensors. This approach enables the use of commodity sensors. Pattern classification provides an image of the overall EMG signal in the entire forearm instead of trying to isolate a high quality signal from specific muscles. This method eliminates the need to search for specific muscles with exact sensor placement. Pattern recognition examines EMG signals from the entire forearm. Studies have suggested that when electrodes are placed around the entire forearm, targeted and untargeted placement of EMG electrodes result in similar classification accuracies. Throughout our experiments, the only effort to position our EMG sensors was placing one of the sensors on the dorsal side of the arm. Even with this untargeted approach, we were still able to use pattern classification with good accuracy. The flexibility in sensor placement means that donning our control unit does not require a therapist, or even a basic understanding of forearm anatomy. For a device that is designed for take-home use in mind, this is an extremely desirable quality. The method also allows for the possibility of an orthosis with more degrees of freedom (DOFs). Current orthoses look at two specific muscles, a flexor and an extensor. The flexor controls the close motion of the orthosis and the extensor controls the open motion. Pattern recognition allows for the recognition of more complex muscle motions, which could control different DOFs of the orthosis.

To acquire the EMG signal, an armband comprising eight EMG sensors and eight IMUs, which can indicate the orientation and acceleration of the device was used. In this iteration of control, we only used the EMG sensors; however, the orientation and acceleration sensors could be useful for future control iterations.

A. Pattern Classification

The pattern classification algorithm seeks to take the eight dimensional raw EMG data from the eight sensors and identify patterns that correspond to certain desired hand motions.

At time t, EMG signals ψt from the sensors was collected and assembled into a data vector

ψₜ:⁻

ψₜ=(e₁^{t} . . . e₈^{t}) (1)

The desired hand state at time t was defined as Ht E {O, C}, where Ht=O corresponds to the intent to open the hand and Ht=C corresponds to the intent to close the hand. While training, ground truth data Hgt is provided by the experimenter who gives the subject verbal commands to open or close the hand. The training period was about 45 seconds—allowing the experimenter to command the user to try to open and then close the hand twice. The first order goal is to predict Ht based on ot. A random forest classifier trained on the ground truth data was used to make this prediction. A random forest classifier is an ensemble machine learning method created from a combination of tree predictors. The trained classifier is then used throughout a complete session as long as the Myo is in the same orientation on the forearm.

We denote the random forest classifier function as:

CLAS(ψₜ)=pₜ^{υ}∈[0,1] (2)

where pOt is the probability that Ht=O (at time t, the user's intention is to open the hand). The converse probability that the user's intent is to close the hand is simply computed as pCt=1−pOt.

B. Output Processing

Raw EMG data of at a rate of 50 Hz was collected. However, the time scale for both hand opening and closing and for pick and place tasks is much lower frequency than the rate at which data is collected, so classifying individual data points correctly is not as crucial as is correctly identifying a hand motion. To identify these motions, we assume that hand posture does not change with high frequency, which allows us to filter and process the probabilities returned by the classifier.

TABLE I
TRAINING PROTOCOL AND ASSIGNED LABELS. FOR EACH
COMBINATION OF INSTRUCTION GIVEN TO THE SUBJECT
AND STATE OF THE EXOTENDON DEVICE, THE TABLE
SHOWS THE GROUND TRUTH LABEL Hₜ^{g} ASSIGNED TO EMG
DATA. TRAINING BEGINS WITH THE TENDON EXTENDED
AND THE SUBJECT ASKED TO RELAX (TOP ROW, MIDDLE
COLUMN) AND PROCEEDS IN COUNTER-CLOCKWISE FASHION.

| | Subject Instruction | | |
|---|---|---|---|
| Device State | Open | Relax | Close |
| Tendon extended | O | C | C |
| Tendon retracting | O | | C |
| Tendon retracted | O | C | C |

While filtering raw EMG signals is a common technique, we chose instead to apply our filter to the results of the classifier. We compute filtered probabilities at time T as:

\hat{p}_{T}^{O}=MEDIAN(pₜ^{O}),t∈[T−0.5 s,T] (3)

\hat{p}_{T}^{C}=MEDIAN(pₜ^{C}),t∈[T−0.5 s,T] (4)

The 0.5 s median filter increases transition delays, but helps eliminate spikes and spurious predictions. We note that, as a result of filtering, generally ̂pOT+̂pC T=˜1.

To produce the final output for our control method, we compare ̂pOT and ̂pC T against two manually set threshold levels, LO and LC respectively. If ̂pOT>LO, then the controller issues an “open” command (retract the tendon). Otherwise, if ̂pC T>LC, then the controller issues a “close” command (extend the tendon). If neither condition is met, no new command is issued and the orthosis simply continues executing the command from the previous step. The values of LO and LC are set manually by the experimenter for each subject after completing training data collection, then kept constant throughout all tests carried out by that subject.

C. Training with the Exotendon Device

The most straightforward method for generating training data to use with the classifier described above would be to simply instruct the user to attempt to open or close the hand, and label the resulting data accordingly. However, we quickly found that this simple procedure is flawed for multiple reasons. First, for stroke patients, we found that the default “relaxed” hand state (attempting to neither open nor close) still produces a strong, subject-specific EMG signal pattern. The classifier would then display a tendency to label this signal as either open or close, unless we provided explicit training data illustrating the difference. Second, we also found that physical interaction with the orthosis itself altered the EMG patterns: for the same user intention, signals recorded with the tendon fully retracted (assisting in hand opening) differed from those recorded with the tendon extended. These issues were addressed through a training protocol and collection of labeled training data.

Specifically, we design our protocol as follows: during training, we instruct the subject to attempt three different hand poses: open, closed, and relaxed. For data collected during both closed and relaxed intents, we assign a ground truth label Hgt=C, corresponding to a closed hand. Since our current target population comprises patients with spasticity, this more closely mimics the subjects' natural state. Furthermore, this means that in order for the orthosis to provide assistance, we must be continuously detecting an active attempt by the user to open their hand. Being conservative in when to send a command to retract the tendon (and thus actively open the hand) reduces the risk of holding the hand open for longer than the subject desires and causing discomfort. We note that one disadvantage is that continuous effort from the subject can lead to muscle fatigue, especially if the subject exerts great strain to provide an open signal.

For all three user intents (open, close, relaxed) we collect training data in different states of the exotendon device, namely with the tendon fully extended, fully retracted, or moving between states. The training procedure is as follows. We start by instructing the subject to relax, with the tendon fully extended. We ask the subject to attempt to open the hand, with the tendon still fully extended. As the subject continues trying to open, the experimenter commands the tendon to retract, opening the hand. One the tendon is fully retracted, we instruct the subject first to relax, then to attempt to close the hand. The experimenter then commands the tendon to extend, allowing the hand to close. Finally, the subject is instructed to relax. This procedure, as well as the ground truth labels assigned at every phase, are summarized in Table I, infra.

The result of this training procedure is a labeled ground truth dataset covering combinations of user intent and device state. We use this dataset to train the classifier described above; finally, at run time, the output of the classifier is used to produce a command for the exotendon device.

### V. EXPERIMENTS AND RESULTS

Testing was performed with 4 stroke survivors, 1 female and 3 male. Subjects were between ages 80 and 39. All subjects showed right side hemiparesis following a stroke event at least 2.5 years prior to the experiment. All subjects had a spasticity level between 1 and 3 on the Modified Ashworth Scale (MAS).

We asked each subject to don the EMG armband and the exotendon device. 4 types of experiments were performed on each subject: 1) EMG control without the device operating: This experiment determined whether the EMG signal present in the hemiparetic forearm arm was strong enough to indicate the subject's intention to open or close. Without the device operating, there was little hand movement, but we still were able to determine the user's intention. 2) EMG control with the device operating: This experiment determined whether EMG control, in conjunction with our exotendon orthosis, could enable hand extension. With the device on, the EMG armband sends raw EMG signals to the classifier, which predicts intent and sends a command based on intent to the motor, which retracts or extends the tendon to move the hand and enable extension. Because this enables extension, it requires the training protocol described above. During this experiment, the subject's forearm was at rest on the table. 3) EMG control during pick and place: This experiment determined whether or not the exotendon device, in conjunction with EMG control, could enable pick and place. The exotendon device enabled hand extension but the forearm was no longer supported by the table. 4) Button control during pick and place: This experiment provided a baseline control comparison for the EMG control. A push button is attached to the device's motor and can be used to retract and extend the tendon. Pushing down and holding the button opens the glove until the hand is fully extended. Releasing the button at any point of the extend cycle causes the tendon to be released immediately and allows the hand to relax. The subject used the button control with the non-affected hand to activate the device and complete pick and place tasks.

Results include two metrics: prediction accuracy and correctly predicted events. Prediction accuracy is defined as the percentage of individual data points ψt predicted by the classifier to be the same as ground truth. However, we believe that the more important metric is the ability to correctly execute a complete, meaningful hand motion, such as opening or closing. We attempt to capture this using the number of correctly predicted events. An event is defined as a change in the user's intention signal, and a correctly predicted event means a predicted event which occurs within 850 ms of the ground truth event, with no incorrect classifications until the next event.

A. EMG Control without the Device Operating

To collect the training set, the subject was asked to try to open and close the hemiparetic hand, with the understanding that the fingers likely would not extend, but that the EMG signal would change as different actions were attempted. The testing set was collected in the same way as the training set. Although the exotendon device was on the hand, the motor was not on. The subject's hand did not move, but the classifier was able to predict the subject's intention by the EMG signals.

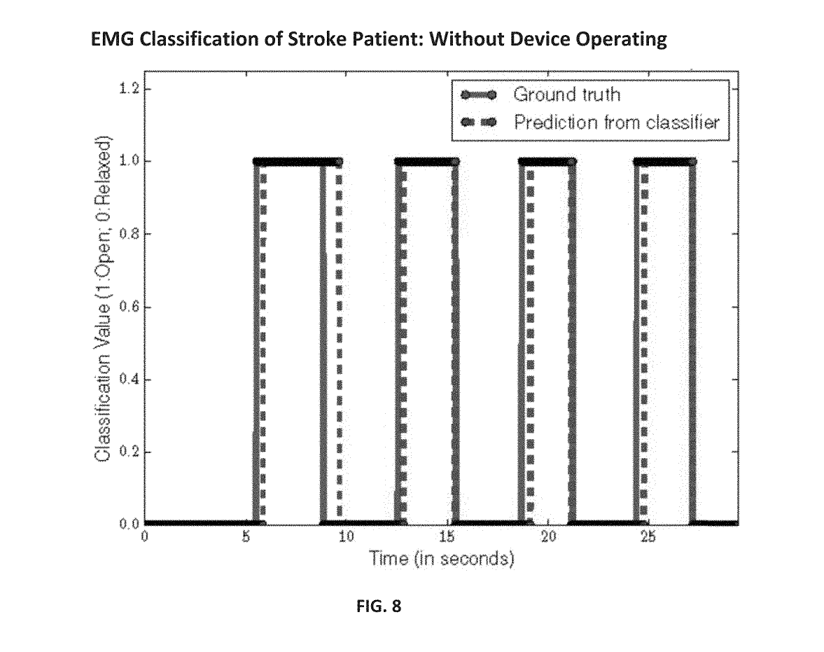

The classifier for Subject A had a prediction accuracy of 85.2% and correctly predicted 11 out of 18 events. The intention for Subject B was predicted with a 90.1% accuracy and 10 out of 16 events were correctly predicted. The classifier for Subject C had a prediction accuracy of 93.6% and correctly predicted 12 out of 14 events. The intention for Subject D was predicted with a 82.2% accuracy and 4 out of 10 events were correctly predicted. An example plot of the ground truth and the prediction results vs. time of Subject A can be found in FIG. 8. See Table II for a summary of the numerical results.

B. EMG Control with the Device Operating

In this section, the device was functioning to open and close the hand, so the training set was collected using the protocol.

The testing set was collected as the subject was asked to try to open and relax the hemiparetic hand while the hand was resting on the table. This time, since the device was operational, when the classifier detected that the subject was attempting to open the hand, the exotendon device would retract the tendon and the subject's hand would extend. If the intention to open was absent, the device allowed the hand to close.

TABLE II
RESULTS FOR NON-FUNCTIONAL MOTIONS

| | |
|---|---|
| Without Device | With Device |

| | | Correct | | Correct |
|---|---|---|---|---|
| Subject | % Accuracy | Events | % Accuracy | Events |
| A | 85.2% | 11/18 | 93.6% | 16/18 |
| B | 90.1% | 10/16 | 83.4% | 4/16 |
| C | 93.6% | 12/14 | 90.9% | 9/11 |
| D | 82.2% | 4/10 | N/A | N/A |

TABLE III
RESULTS FOR FUNCTIONAL MOTIONS (PICK AND PLACE)

| | Correct Events - | Correct Events - |
|---|---|---|
| Subject | EMG Control | Button Control |
| A | 6/13 | 3/3 |
| C | 6/6 | 5/5 |

Subject D was not included in these results because of subject fatigue. The classifier for Subject A had a prediction accuracy of 93.6% and correctly predicted 16 out of 18 events. The classifier for Subject B had a prediction accuracy of 83.4% and correctly predicted 4 out of 16 events. Subject

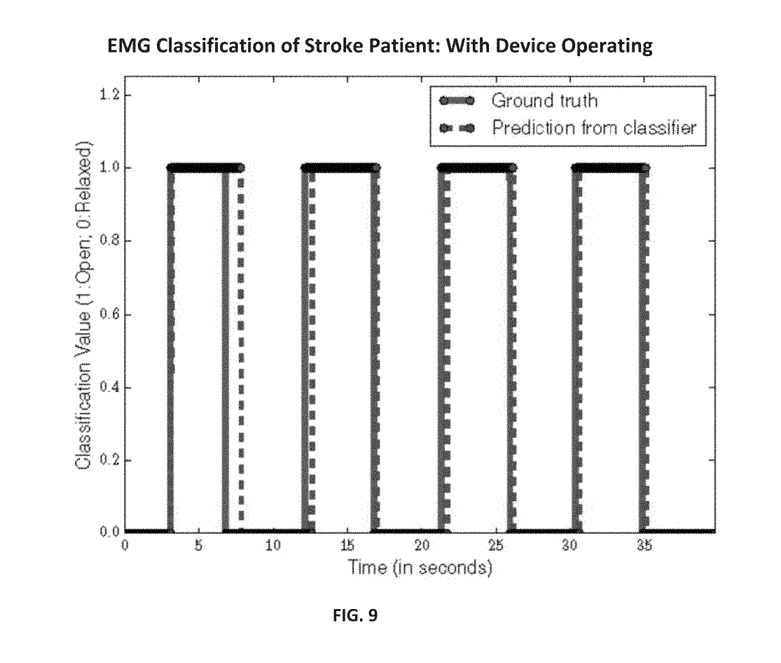

C had a percent accuracy of 90.9% and the classifier correctly identified 9 out of 11 events. Subject B had a MAS spasticity level of 3 in her finger flexors, while Subjects A and C had a MAS spasticity level of 2 in these joints. This is a possible explanation for why Subject C's percent accuracy and correct event prediction are lower. An example plot of the ground truth and the prediction results vs. time of Subject A can be found in FIG. 9. See Table II for a summary of these results.

C. EMG Control During Pick and Place

Precise ground truth is difficult to establish when the subject is performing pick and place tasks because an operator instructing the user when to begin and end extension would result in unintuitive grasping. Instead of percent accuracy, we instead use the number of correctly executed pick and place tasks as a metric for the pick and place experiments (both with EMG control and with button control).

The training for this experiment was the same training as for the experiments with EMG control with the device operating. We did not do additional training for this set, but used the classifier from the previous experiment to control the pick and place.

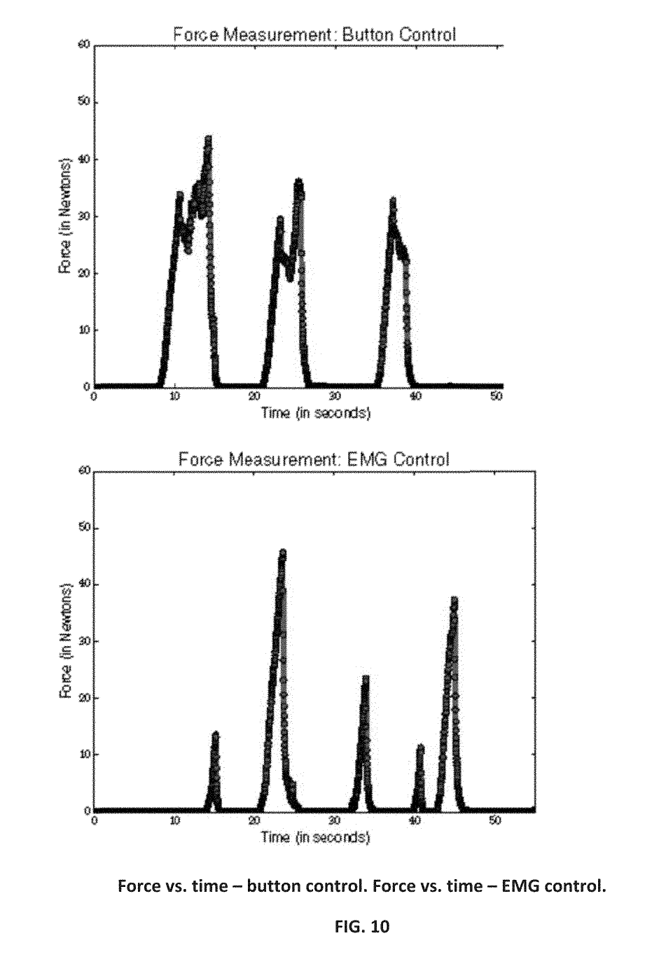

During testing, an object was placed in front of the subject and they were asked to operate the exotendon device using EMG control in order to pick the object up, move it several inches, and then place it back down. The details of a complete pick and place motion, as well as the exotendon's role in the action are described in FIG. 10.

Subject B was not included in the results for pick and place because sizing issues with the exotendon device rendered her unable to grasp the objects. Because of subject fatigue, Subject D was also not included. Subject A successfully completed 6 out of 13 pick and place attempts. Subject C completed 6 out of 6 pick and place movements. We note that Subject C was higher functioning than the other subjects and was generally able to complete an unassisted hand extension, albeit with significant difficulty. Nevertheless, the subject reported that the exotendon device provided assistance in hand opening during pick and place. See Table III for a summary of these results.

D. Button Control During Pick and Place

Before testing, the subject was instructed on how to control the device using their left hand, and then allowed to try the control button for several minutes before being asked to perform pick and place tasks. During testing, the subject was asked to pick and place the same object as they had during the EMG controlled pick and place.

Again, Subjects B and D were not included in these experimental results. Subject A successfully completed 3 out of 3 pick and place attempts. Subject C successfully completed 5 out of 5 pick and place attempts with the button control. See Table III for a summary of these results.

E. Tendon Forces During Pick and Place Experiments

To assess the ability of the exotendon device to correctly interpret user intent, as well as the level of discomfort caused by operation, we measure the forces applied on the exotendon network during pick and place experiments. For the same subject, we compared peak forces obtained for EMG control versus button control. Our assumption was that an incorrect interpretation of a “close” signal, where the subject intended to close the hand but the assistive device did not react appropriately, would show up and a spike in the force levels as the subject would be effectively fighting against the orthosis.

During the button controlled pick and place, the peak force on the tendon was 53.7N for Subject A and 58.6N for Subject C. During the EMG controlled pick and place, the peak force on the tendon was 59.6N for Subject A and 77.6N for Subject C. An example plot of the force vs. time during the EMG controlled experiment and during the button controlled experiment can be found in FIG. 5; each time series contains multiple hand extensions. We measured all forces on the using a load cell (Futek, FSH00097) placed in series with the exotendon network. The peak force for the two controls thus showed little difference for Subject A, but for Subject C, the EMG control lead to higher forces. However, there was enough variance between force peaks obtained with the same control mechanism to suggest that the difference could fall within normal operating range. Additional testing will be needed to further study this issue.

Overall, the results showed effective pattern classification performance, to the level of physically enabling functional hand motion. Most of the incorrectly predicted events were the result of the control not correctly recognizing the change in intention within the allowed 850 ms window, rather than spikes caused by misclassification in the middle of the event. These delays were caused in part by the median filter, which operates the past 500 ms to inform the control, thereby adding lag. Another possible cause was subject spasticity, which made it difficult for the subjects to relax or close after activating their extensor muscles. The results were trained and tested on separate data sets, both of which were taken from the same patient during the same session.

Pick and place experiments controlled by EMG generally showed lower accuracy than non-pick and place experiments where the device was operating. The difference between the 2 types of experiments was that in the former the subject's arm was engaged in the task, while in the latter the forearm was simply resting on the table. We hypothesize that, because of the abnormal coactivation present in stroke subjects, a trained classifier which was trained while the forearm was resting on the table is confused by extension of the arm during a functional grasping motion.

As our work is eventually intended as a take-home device, the level of automation of the training is an important consideration. In this iteration of our work, an operator was required to provide the training set with ground truth while instructing the patient to try to open or close. The operator also used the button control to implement the training protocol when required. In the future, both of the above responsibilities could easily be transferred to a graphical user interface using visual cues instead of verbal commands, instructing the patient when to try to open and close, and programmed motor actions.

It has been shown that an EMG based pattern classification control of an exotendon device can enable functional movement in a stroke survivor. The control achieves high accuracy during non-functional open and close hand motions, and can enable functional motions, like pick and place. The pattern classification technique allows the use of commodity devices which are easy to don, as there is no need to place sensors on specific muscles. The control is intuitive and does not require an extended period of training. Thus, the devices of the described embodiments permit functional movement enabled by EMG control in wearable devices.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A functional hand orthotic comprising:
a flexible glove having a dorsal surface and a palmar surface and a plurality of sheaths configured to receive digits of a user's hand,
a network of exotendons disposed on a surface of the glove, the network of exotendons comprising a plurality of exotendons,
wherein the network of exotendons is operatively engaged to one actuator, the one actuator capable of actuating multiple exotendons simultaneously; and
a controller configured to receive a plurality of input signals from a plurality of sensors placed around the circumference of the user's forearm, determine the user's intention to execute a hand movement based on the plurality of input signals according to a pattern classification algorithm, and send a command signal to the actuator to actuate at least one of the plurality of exotendons.

2. The functional hand orthotic of claim 1, wherein the exotendons are routed on the dorsal surface of the glove.

3. The functional hand orthotic of claim 1, wherein the network of exotendons has five exotendons.

4. The functional hand orthotic of claim 1, wherein the network of exotendons has four exotendons.

5. The functional hand orthotic of claim 1, wherein the network of exotendons has three exotendons.

6. The functional hand orthotic of claim 1, wherein the network of exotendons has two exotendons.

7. The functional hand orthotic of claim 5, wherein each of the three exotendons is routed along a different sheath of the glove.

8. The functional hand orthotic of claim 7, wherein the first exotendon is routed along a sheath that corresponds to an index finger of the glove, the second exotendon is routed along a sheath that corresponds to a middle finger of the glove, and the third exotendon is routed along a sheath that corresponds to a ring finger of the glove.

9. The functional hand orthotic of claim 8, wherein the first second and third exotendons terminate at a junction.

10. The functional hand orthotic of claim 9, wherein the junction is proximate the actuator.

11. The functional hand orthotic of claim 1, wherein the glove is formed from washable fabric.

12. The functional hand orthotic of claim 1, wherein the glove further includes a sleeve wearable on the forearm of a person.

13. The functional hand orthotic of claim 12, wherein the sleeve is detachable from the rest of the glove.

14. The functional hand orthotic of claim 13, wherein the actuator is mounted on the sleeve.

15. The functional hand orthotic of claim 1, wherein the actuator is an electric motor.

16. The functional hand orthotic of claim 15, wherein the motor weighs no more than about 40 g.

17. A functional hand orthotic comprising:
a flexible glove having a dorsal surface and a palmar surface and a plurality of sheaths for receiving digits of a user's hand,
a first plurality of exotendons disposed on the dorsal surface of the glove, a second plurality of exotendons connected to the first plurality of exotendons, the second plurality of exotendons disposed on the palmar surface of the glove, and
a wearable single actuator operatively engaged to the first and second pluralities of exotendons, wherein the first and second plurality of exotendons are actuated by the single actuator; and a controller configured to receive a plurality of input signals from a plurality of sensors placed around the circumference of the user's forearm, determine the user's intention to execute a hand movement based on the plurality of input signals according to a pattern classification algorithm, and send a command signal to the actuator to actuate at least one of the plurality of exotendons.

18. The functional hand orthotic of claim 17, wherein each of the exotendons is routed along a different sheath.

19. The functional hand orthotic of claim 18, wherein the exotendon is routed along the dorsal side of one sheath and traverses to the palmar side of the glove.

20. The functional hand orthotic of claim 19, wherein the first plurality of exotendons is routed along the dorsal side of the glove and traverses to the palmar side of the glove.

21. The functional hand orthotic of claim 20, wherein the first plurality of exotendons is routed along the dorsal side of the glove and traverses to the palmar side of the glove to define the second plurality of exotendons.

22. The functional hand orthotic of claim 19, wherein the exotendon extends from the proximal end of the sheath proximally along the metacarpal section of the glove.

23. The functional hand orthotic of claim 17, wherein the plurality of exotendons are on the palmar surface of the glove and flexion side of the MCP joint.

24. The functional hand orthotic of claim 22, wherein the plurality of exotendons traverses to the dorsal side of the PIP and DIP joints.

25. The functional hand orthotic of claim 24, wherein the orthotic enables a fingertip pinch grasp.

26. The functional hand orthotic of claim 17, wherein the actuator allows both grasping and extension movements of the digits when worn by a user.

27. A functional hand orthotic comprising:
a flexible glove having a dorsal surface and a palmar surface and a plurality of sheaths configured to receive digits of a user's hand,
a network of exotendons;
an actuator operatively connected to the network of exotendon, wherein the network of exotendons includes exotendons having a longitudinal body routed on the palmar side of the glove and flexion side of a MCP joint of a user, and further wherein the exotendons' longitudinal body traverses to the dorsal side of the PIP and DIP joints of the user to enable a fingertip pinch grasp; and
a controller configured to receive a plurality of input signals from a plurality of sensors placed around the circumference of the user's forearm, determine the user's intention to execute a hand movement based on the plurality of input signals according to a pattern classification algorithm, and send a command signal to the actuator to actuate at least one of the plurality of exotendons.
